(19) United States
(12) Patent Application Publication
(10) Pub. No.: US 2003/0153947 A1
(43) Pub. Date: Aug. 14, 2003

(54) Sternum suture material and its manufacturing method

(76) Inventor: Tomoaki Koseki, Chiyoda-ku (JP)

Correspondence Address: Tomoaki Koseki, 17-2, Sotokanda 2-chome, Chiyoda-Ku, Tokyo, 101-0021, JP

(21) Appl. No.: 10/358,049

(22) Filed: Feb. 3, 2003

(30) Foreign Application Priority Data
Feb. 14, 2002 (JP) JP 2002-036480

(51) Int. Cl. A61L 17/00

(52) U.S. Cl. 606/228

(57) ABSTRACT

Monofilament wires or threads are woven, becoming a wire with ring forms. The center part of the wire is in a plain weave in a tape form. The material is to be either non-magnetic property material or bio-absorbable material. An automatic weaving machine is utilized to manufacture the wire. The center part of the metal cable is woven with thin metal filaments, and either one end or both ends of the cable are hardened with silver wax to facilitate the attachment of a suture needle to the ends of the cable. Also, the hardened end of the cable is directly processed into a suture needle form. A U-shaped sternum fixing hook and an adjusting screw press and fix the sternum, a piercing tool pierces a hole into the sternum, then sternum suture material is passed through the sternum, then the sternum suture is conducted. The thickness of the base of the sternum fixing hook is set to prevent the needlepoint of the piercing tool from reaching the lung, or a protection plate can be provided. The center part of the wire is made in a U-shape in advance. A straight needle with a wire is fixed, and pierced from below the sternum. Sternum fixing hooks are continuously fixed with ball joints, making a fixing arm and a movable arm, having the function to open and close the incision sides of the sternum. With the above proposal it is possible to provide a smooth and easy-to-handle sternum suture material which does not establish metal fatigue, prevents halation when using the MRI, health insurance can be applied, easier to fasten, less bleeding, prevents vertical gaps of the sternum when closing the sternum. Piercing the sternum can be done easily, precisely, and safely, and then conduct the sternum suture. The operator does not need to remove needles or guide-wires. This is less invasive to the sternum. This invention does not harm the lung. An affordable and convenient manufacturing method can be provided.

### BACKGROUND OF THE INVENTION

1. Field of the Invention

This invention relates to sternum suture materials and tools utilized during cardiac surgery.

2. Description of the Prior Art

Sternum is located at the center of the chest, to which the ends of the ribs are connected. During cardiac surgery, the center of the sternum is incised, and the sternum is opened to the right and to the left to conduct the surgical operation. The wire usually used after the surgery for closing the sternum is monofilament made of metal. The current standard methods of usage of such wire are; a needle type wherein a curved needle to pierce the sternum is attached to the end of the wire, the curved needle is pierced from the top of the sternum to the bottom, then the needle is pulled up on the other side from the bottom to the top; another form of needle type is using curved needles attached to both ends of the wire and the sternum is pierced from the bottom to the top on both sides. A different method is a gimlet type wherein thin guide-wires are attached to both ends of a thick wire, the sternum is pierced from the top using a gimlet that has a hole at the tip, the guide-wire is threaded through the hole and is pulled from the bottom. In the case of the needle type the two wire ends that come out above the sternum at both the right and the left sides, the needle is cut with pliers, and in the case of the gimlet type the two ends of the guide-wire are cut with pliers, and then in both cases the ends are grasped with a pair of specific forceps and then fastening is conducted.

In some facilities, needles are attached to polyester thread for the above usage.

The material of sternum suture wire is stainless steel SUS316L, and currently there are no commercialized product made of non-magnetic substance as the material. Also majority of the current sternum suture materials is made of monofilament wire, and metal cables are not commonly utilized.

The outside of the sternum is covered with hard cortical bone, but the inside is weak and it is easy to bleed. Furthermore, many cardiac patients are elderly people whose sternum is frail, so when piercing is conducted forcefully using dull-ended curved needle, fractures of the sternum often occurs.

The intensity against metal fatigue of monofilament metal wire is weak within the body. The chest part especially tends to receive burden from metal fatigue due to respiration, coughing, sneezing and such.

In a case where the sternum suture wire material is stainless steel, halation occurs when using Magnetic Resonance Imaging after the surgery, and the displayed picture becomes disordered.

When utilizing polyester thread, health insurance cannot be applied as the thread is not a specific insured medical material. Also as metal is not used, electric hemostatic method cannot be conducted using electric scalpel. There occur gaps in the sternum in a vertical direction after the suture.

One proposal is to utilize a gimlet that has a hole at the needlepoint, the gimlet is pierced from directly above the sternum, the wire is hooked from the inside of the sternum and pulled up, but the procedure of threading through a thin wire into a tiny hole during surgery is cumbersome for the operator.

In cases of utilizing needles at either one end or both ends of the wire, needles being thicker than the wire, there occur differences between the wire and the needle, which causes much bleeding from the needle hole, and that takes time and effort for the hemostasis procedure.

Metal wires have weak ligation function, and especially pure titanium wire has weak twisting feature. When suture of sternum is conducted with a material with its cross-section being a columnar form, the left and the right sides of the sternum tends to cause gaps in the vertical direction after the fastening.

Monofilament wire is hard and difficult to handle. When needles are to be attached onto flexible metal cables, the cable-ends tend to become loose and attaching the needle is difficult.

As monofilament is hard, it often damages fragile sternum. When a needle is attached to a monofilament, there occur differences between the needle and the filament that causes bleeding when suturing of the sternum.

When using the needle type method, the curved needle is grasped and pierced with a needle-holder, therefore the needle point does not go through the sternum, a procedure of which is a hard difficult one for the operator. If the needle is forcefully inserted, there are cases where the sternum fractures. As the needle is thick and curved, there occur gaps between the pierce hole and the diameter of the suture material, which causes bleeding and it would take time and effort on the part of the operator for hemostasis. Also the operator would have to cut the needle with pliers at the time of the fastening.

In the case of gimlet type, damage on the lung becomes a risk as the gimlet is pierced from the top of the sternum to the bottom of the sternum. Sometimes the operator would have to use spoons and such to protect the lung. Once a hole is made with the gimlet, and when the wire is to be inserted directly into the hole after taking out the gimlet, the surrounding tissue covers the hole and the position of the hole cannot be confirmed. The operator would have to cut the guide-wire with pliers at the time of fastening.

### SUMMARY OF THIS INVENTION

To solve the above issues, this invention proposes the following. Thin monofilament wires are woven to make one loop form or a multiple chain-like cable, the woven wire becomes a straight line with tension during suture, and it is easy to fasten the wire utilizing the ring loop or the chain-like cable. Either both ends or one end of the wire has a straight needle or a curved needle connected, the needle with which to pierce the sternum, wherein the tip ends of the wire to which needles are connected are made thinner compared to the center part, to avoid any gaps or differences between the needle diameter and the wire connection part.

The thread or the needle material is of non-magnetic property, or a material that can be absorbed within the body.

The manufacturing method herein adopted is, when weaving the thread or the wire, an automatic weaving tool Torsion is used to enable uniform continuous weaving, to make one loop or a multiple number of rings forming a chain-like cable, the ends of the wire being thinner than the center part, a hole is drilled in a vertical direction at the bottom end of the needle with laser or discharge processing, the hole to which the thinly narrowed end of the wire is inserted, thence the circumference of the needle is uniformly compressed to fix the needle and the wire without any gap or difference.

The manufacturing method herein adopted is, when weaving the thread, a braiding machine is used to enable uniform continuous weaving, to make one loop or a multiple number of chain-like cable, wherein the ends of the wire being thinner than the center part, a hole is drilled in a vertical direction at the bottom end of the needle with laser or discharge processing, the hole to which the thinly narrowed end of the wire is inserted, thence the circumference of the needle is uniformly compressed to fix the needle and the wire without any gap or difference.

After fastening, the center part of the wire that would be located within the sternum is made of plain weave, a form of which would prevent any vertical gap on the sternum. The ends of the wire are thinly narrowed, and the straight needle or a curved needle for piercing the sternum are connected to either one end or both ends of the wire.

Using the braiding machine or the Torsion, the center of the cable has thin metal fiber woven in and made into a thread form, and one or both ends of the cable has flax applied, to which silver wax is adhered by heating with a burner, to make the ends hard and solid to enable the suture needle to be easily attached.

Using the braiding machine or the Torsion, the center of the cable has thin metal fiber woven in and made into a thread form, and one or both ends of the cable has thin metal lines fixed and hardened by waxing, and then processed into suture needle in either straight form or curved form utilizing a pressing machine. The tip end of the needle is made as a dull end.

Using the braiding machine or the Torsion, the center of the cable has thin metal fiber woven in and made into a thread form, and one or both ends of the cable has flax applied, to which silver wax is adhered by heating with a burner, to make the thin metal fibers to be adhered hard and solid, and then processed into suture needle in either straight form or curved form utilizing a pressing machine.

This invention also proposes a set of sternum piercing tool consisting of three parts, being the sternum fixing hook, adjusting screw, and piercing tool, wherein an adjusting screw connected to the upper part of a U-shaped sternum fixing hook is rotated, the sternum is pressed and fixed. A hole for the piercing tool is made in the center of the adjusting screw in a vertical direction. There is a hole in the lower part of the sternum fixing hook in the direction of the needlepoint of the piercing tool edge, and the hole is continued into the side slit to extract the sternum suture material after being passed through the sternum. The sternum is pierced with the piercing tool from the hole at the top part of the adjusting screw, the sternum suture material is threaded through to the sternum, and then the sternum suture operation is conducted.

The sternum fixing hook has a vertical hole on its back side in the center, the columnar shaft is inserted to that hole, and the columnar shaft is continued on to an elevation table. The elevation table is united with the piercing tool grip part sandwiched with a fixing plate. The columnar shaft is made to have the function as the guide, when the elevation table is rotated having the columnar shaft as the axis, the needlepoint of the piercing tool is matched with the position of the hole, when the piercing tool is lowered down.

The thickness of the lower part of the sternum fixing hook is designed so that the needlepoint of the piercing tool digs into the top end of the base side of the sternum fixing hook when the piercing tool is pierced into the sternum in cases where the height of the inside of the sternum fixing hook is the same as the thickness of the sternum, and so that the piercing tool needlepoint does not go beyond the base of the lower part of the sternum fixing hook when the piercing tool needlepoint is pierced into the sternum in cases where the sternum is thin after screwing in the adjusting screw to its maximum.

The sternum fixing hook is structured so that the lower hole part is projected to expand at the lower end like a cone shape, wherein the piercing hole is at the center of the cone, and thence the sternum suture material can be passed through easily.

The sternum fixing hook is structured so that a plate is continued from the lower part to the front side or to the back side vertically downwards, and the plate is bent perpendicularly once again to be in parallel to the lower part of the sternum fixing hook, to function as the protection plate to protect the lung from being damaged by the needlepoint of the piercing tool.

The sternum suture wire is structured to have the center part to be bent in advance in a U-shape, the length of the center of that bent portion is equal to the distance between the two holes made on the right and the left sides of the sternum using the sternum suture material, and the two ends of the wire are inserted from below the sternum out from the upper part of the sternum to be fastened.

The structure of the sternum piercing tool is that the two ends of the piercing tool are united with the connected suture materials. The part connecting the piercing tool and the suture material is pressed within the piercing tool fixing slit at the lower part of the sternum fixing hook, and then screwed in and fixed from the side with a fixing screw. A thread cutting hole in which the pull-up-screw to pull up the material is inserted is provided at the upper part of the sternum fixing hook, and the lower end of the pull-up-screw has a hole wherein the needlepoint of the piercing tool can be inserted. When the pull-up-screw is rotated, the sternum fixing hook is pulled up, the piercing tool pierces through the sternum from the back side, and the needlepoint of the piercing tool digs into the lower end of the pull-up-screw. Then the fixing screw is unscrewed, the sternum fixing hook is taken away, the needlepoint of the piercing tool is pulled up above the sternum to conduct the suture.

The sternum suture wire has the following features. A hole is made with a drill, discharge processing or laser to provide a hole in a vertical direction at the needlepoint of the straight needle having an appropriate length to pierce through the sternum. The needle is inserted to either one end or both ends of the wire, and connected with caulking processing to prevent gaps or differences between the needle circumference and the wire. The cross-sectional form of the needle is made to be in a triangular or square form using press processing, to facilitate the needle to pierce through even hard sternum. The material of the wire is soft quality metal material aiming for less invasiveness and damage onto the sternum.

The sternum fixing hook has the following structure. A ball joint is applied to the upper side of the sternum fixing hook, a multiple number of sternum fixing hooks are fixed continuously, forming a fixing arm and a movable arm to apply to the incision side of the sternum when cutting open the sternum to the right and left, and thus the sternum incision sides open and closes by moving the two arms in a parallel manner.

### DESCRIPTION OF THE PREFERRED EMBODIMENTS

Hereafter, a description of the preferred embodiments of this invention is made in reference to drawings. FIG. 1 shows a sternum suture wire according to claim 1, wherein monofilaments are woven to form a chain-like wire with multiple small rings within. The wire part 1 becomes a straight line during suture operation due to tension, and the form is convenient enabling easy fastening utilizing the rings of the wire. A curved needle 2 to pierce through the sternum is connected to one end of the wire. The wire end 3 to which the needle is connected is woven in a thinly narrowed way from the center part, enabling almost the same diameter for both the needle and the wire to prevent bleeding. Usually, the material for a sternum suture wire is made of SUS316L with high bio-compatibility, but when using the Magnetic Resonance Imaging device after the surgery, it is significant to use non-magnetic material titanium according to claim 3. The center part of the wire 1 has the diameter of 0.8 mm which is the most common size of a wire used for an adult's surgery. The tip end of the needle has a hole made in vertical direction with the diameter of approximately 1.5 mm to 2.0 mm made with a drill, laser or a discharge process, the end of the wire is inserted into that hole in the needle, the circumference of the needle is uniformly compressed, to fix the needle and the wire. After the suture, the needle is cut off, and the chain rings of the wire are used for repeated return-sewing suture and then fastening.

FIG. 3 shows the sternum suture material according to claim 2 wherein the center part of the wire 4 is made in plain weave in a tape form. The cross-section of the center part 4 is in an oval form that increases the fastening intensity and prevents any vertical gaps to occur on the sternum. A curved needle 5 to pierce through the sternum is connected to one end of the wire. Wire end 6 is woven into a thinly narrowed form to prevent bleeding from the needle hole. When utilizing the material according to claim 4, there occurs no halation when using Magnetic Resonance Imaging device, and as it will be absorbed within the body, there will be no foreign substance to be left in the body. Bio absorbable material are polyglycolic acid, polylactic acid, polydiauxanon, policaprolacton and such.

FIG. 5 shows one embodiment of a cable end 9 of a metal cable 8 manufactured with the manufacturing method according to claim 7. Flax is applied to the cable ends, silver wax is adhered by heating with a burner, to harden the cable end to prevent loosening of each thin metal filament, thus facilitating the attaching of the needle for suture.

FIG. 6 shows one embodiment of metal cable 11 with one of its ends becoming a needle 10, to directly pierce through the sternum. The center of cable 11 is made in a thread form woven by using a braiding machine or a Torsion, and flax is applied to one end of the cable, thin metal fibers are adhered and hardened after silver wax is applied an heated with a burner, and then processed into a curved form suture needle 10 using a pressing machine.

FIG. 7 shows an embodiment of a sternum hole piercing tool according to claim 10 and claim 12. A thread cutting hole is made on the upper part of a U-shaped sternum fixing hook 12 into which an adjusting screw 13 is inserted, and when the adjusting screw 13 is rotated and screwed, the base side of the sternum fixing hook 12 and the adjusting screw 13 presses and fixes the sternum. On the top of the base side of sternum fixing hook 12, there are slip-proof spikes 19 provided on four corners. A hole for the piercing tool 14 is made in the center in a vertical direction of adjusting screw 13. A hole is made on the lower part of sternum fixing hook 12 in the direction of the needlepoint of piercing tool 14, and that hole is connected with side slit 15 which is to pull out the sternum suture material through the sternum fixing hook 12 after the material are let through the sternum. A hole is pierced into the sternum by rotating the piercing tool 12 through the hole at the upper part of the adjusting screw 13. The needlepoint of piercing tool 14 would have the best sharpness if the tip is in a drill form, but a gimlet form or a needle form are acceptable. The figure shows an embodiment to show that the thickness of the sternum fixing hook 12 is designed in such a way that when the sternum is thin, the adjusting screw 13 is screwed in to its maximum, but the needlepoint of the piercing tool 14 after pierced into the sternum does not go beyond the base side of the sternum fixing hook 12. In cases when the sternum is thick, the adjusting screw 13 can be moved upward. When the base side of the adjusting screw 13 is in the same position as the base side of the sternum fixing hook 12, the needlepoint of the piercing tool 14 digs into the upper side of the lower part of the sternum fixing hook 12. Therefore, for any thickness of sternum, the needlepoint of the piercing tool 14 would pierce the sternum without reaching the lung. After a hole is pierced, the piercing tool 14 is pulled out, the suture material such as the wire are passed through from the hole of adjusting screw 13 to the lower part of sternum fixing hook 12, while the sternum fixing hook is still fixed. The passing through of the material can be conducted from the bottom to the top. The suture material is pulled out through slit 15, pulled up to above the sternum, and then the sternum fixing hook 12 is taken off from the suture material. No guide-wires are necessary to treat the said suture material and needles, enabling the total cost to be relatively achievable. The material for these tools can be made of metal such as stainless, but the material except for the needlepoint of the piercing tool 14 can be resin and there are no problem regarding the strength.

FIG. 8 shows one embodiment of a sternum piercing tool according to claim 11. A hole is made in a vertical direction at the back side center of sternum fixing hook 12, a columnar shaft 20 is inserted into that hole, and the end of the columnar shaft 20 that is projected above is bent perpendicularly and is continued with elevation table 18. The elevation table 18 is in a united form being pressed between the grip of piercing tool 14 and the fixing plate 21, the elevation table 18 is rotated having the columnar shaft 20 as the axis, the needlepoint of the piercing tool 14 is matched with the position of the hole to be pierced, and when the piercing tool 14 is pressed upward, the columnar shaft 20 acts as the guide. The upper part of the base side of the sternum fixing hook has some ditches applied as a slip proof process. The base side of the lower part of the sternum fixing hook expands outside as in a cone shape, and the lower part becomes the piercing hole 17, having a structure to facilitate the passing through of the sternum suture material from the bottom.

FIG. 9 shows a sternum piercing tool according to claim 14. A plate is stretched from the front side of the lower part of the sternum fixing hook 12 continuously in a vertical direction downward, and then the plate is bent once again perpendicularly to be in parallel with the base side of the sternum fixing hook 12, then the plate becomes the protection plate 16 to protect the lung from any possible damage from the needlepoint of piercing tool 14. The piercing tool is in a form of a wire with a straight needle, and that is effective when piercing is desirable to be conducted from top to bottom.

FIG. 10 shows a sternum suture wire according to claim 15, the wire of which is to be used for suture of sternum after making a hole with a sternum piercing tool. The center part of the wire is bent in U-shape in advance, and the length of the bent part is equal to the distance between the two holes on the sternum, one on the right and the other on the left, both holes pierced with the sternum piercing tool. Both ends of the wire is inserted into the sternum from the bottom to the top, and then fastened. When fastening, the bent central part of the wire attaches to the sternum inside the sternum and prevents loosening of the wire. The material suitable for the wire is SUS316L in the case of stainless steel, and high-purity titanium in the case of titanium especially considering the bio-compatibility and intensity.

FIG. 11 shows an embodiment of a sternum piercing tool and wire with a straight needle attached to one end according to claims 16 and 17. A wire to which a straight need 25 is attached is pressed inside the piercing tool fixing slit 24 at the base part of the sternum fixing hook 12, and the wire is fixed from the side after screwed by fixing screw 23. A thread cutting hole is made at the top part of the sternum fixing hook 12 in which a pull-up-screw 22 is to be inserted, and there is another hole at the base of the pull-up-screw 22 in which the needlepoint of straight needle 25 is made. When the pull-up-screw 22 is rotated, the sternum fixing hook 12 is pulled up, the straight needle pierces the sternum from the backside, and the needlepoint digs into the base of the pull-up-screw 22. Then the fixing screw 23 is loosened, the sternum fixing hook 12 is taken off, the needlepoint of the straight needle is pulled up above the sternum and then the suture is conducted. The length of the straight needle is in the appropriate length to pierce through the sternum when an operator holds one end of the needle, thus approximately 30 mm to 45 mm. A hole is made in a vertical direction at the tip of the needle with discharge processing, the needle is inserted into the end of the wire, and the needle and the wire are connected with caulking processing to prevent any gaps at the circumference. The cross-section of the needle is in a triangular form made with press processing to facilitate the piercing of the needle even through hard sternum. The material of the wire is soft quality stainless steel SUS316L which is less invasive to the sternum, or in a case of titanium, high purity titanium is appropriate regarding its bio-compatibility and intensity.

FIG. 12 shows a sternum piercing tool according to claim 18, that has the function to open and close the incised sternum. A ball-joint is attached to the top side of sternum fixing hook 12, then connect a multiple number of sternum fixing hooks 12 continuously so that the hooks would move flexibly. These hooks act as fixing arm 26 and movable arm 27 to apply to the incision side of the sternum when the right and the left sides of the sternum are opened, one end of the fixing arm 26 is connected with one end of rail 29 perpendicularly, one end of movable arm 27 is in a box structure to wrap around the rail 29. Ditches are applied continuously to the sides of the rail, there are two pins inside the box to move the movable arm 27, the pins fit into the rail ditches, then connected to handle 28 which is projected above the box, and when handle 28 is rotated the movable arm 29 moves along the rail 29, and then opens and closes the incised sternum. As this tool presses and opens the incised side of the sternum directly, there is no need adjust and fix the sternum according to the thickness of the sternum. As sufficient number of sternum fixing hook 12 are connected to suffice the number of fastening, the operator does not have to thread through the suture material by moving each piece one by one. As the burden can be diffused by having many joints, it is possible to have the material of the whole parts to be disposable made of resin and such material instead of metal.

This invention is enforced in the above embodiments, and has the following effects.

As this is structured having thin wires woven in, this is durable against metal fatigue within the body.

By utilizing non-magnetic material for the sternum suture wire, the MRI display pictures do not cause any halation, therefore clear pictures can be shown enabling appropriate diagnosis.

In a case when metal wire is used, medical insurance can be applied as the metal wire is designated as a the specific insured medical material. It is possible to conduct hemostasis using electric scalpel by charging electricity. No gaps occur in vertical direction of the sternum after suture.

In the case of a wire with a needle at the end, the surgery can be conducted more smoothly compared to current tools and is convenient for the operator.

As there are not much differences between the needle circumference and the wire, bleeding would not occur from the needle hole. That would not cause extra time or effort on the part of the operator for hemostasis.

As fastening is conducted by utilizing the chain-like ring parts of the wire, fastening is easy and more firm compared to the conventional wire-to-wire fastening. Even when titanium is used as the material, there is no problem from lack of twisting force.

By utilizing an automatic weaving machine or Torsion in the manufacturing process, it is possible to supply uniformly made affordable sternum suture thread, contributing to the cost reduction of medical expense.

As the center part of the wire is made of plain weave in a tape form, the cross-section of the wire is oval, which increases the fastening intensity, and would prevent vertical gaps to occur in the sternum.

With this invention, it is possible to use a soft and easy-to-handle metal cable as the sternum suture material, with which there will be less invasion onto the sternum, and when the end of the wire is processed directly into a needle form, there will be no gap between the needle and the wire, which decreases the risk of bleeding.

As the needlepoint of the needle is made in a dull-end form, there is less concern of damaging the lung and such tissues when a cable is passed through after making a base hole into the sternum with drills and such.

This invention enables an easy, precise, and safe piercing of a hole into the sternum, and for passing the suture material through the sternum. There is no difference between the diameter of the pierce hole and the diameter of the suture material, which decreases the risk of bleeding. In situations according to claims 1, 2, 3, 4, and 9, the operator does not have to cut the needle or a guide-wire with pliers during fastening. There is no concern of the needlepoint of the piercing tool to reach the lung and make any damages.

The thoracotomy sternum piercing tool according to claim 9 does not need to be fixed in each occasion according to the thickness of the sternum as the tool presses and opens the sternum incision sides directly. As a multiple number of sternum fixing hooks sufficient according to the number of the fastening is already connected in advance, the operator does not need to pass through the suture material one by one by moving each hook. Also, as there are many joints, the form fits the form of the sternum, so there will be less burden on the patient, and therefore there is less concern of pain in the back bone after surgery.

### BRIEF DESCRIPTION OF THE DRAWINGS

FIG. 1 is an external drawing of a sternum suture wire with a needle connected to the end.

FIG. 2 is an external drawing of the sternum suture wire during usage.

FIG. 3 is an external drawing of a sternum suture wire with a needle connected to the end.

FIG. 4 is an external drawing of the sternum suture wire during usage.

FIG. 5 is an external drawing of a metal cable wherein one end is hardened.

FIG. 6 is an external drawing of a metal cable wherein one end of the cable is processed into a needle form.

FIG. 7 is an external drawing of a sternum piercing tool.

FIG. 8 is an external drawing of a sternum piercing tool with an elevation table.

FIG. 9 is an external drawing of a sternum piercing tool with a protection plate attached.

FIG. 10 is an external drawing of a sternum suture wire.

FIG. 11 is an external drawing of a sternum piercing tool to which a straight needle is fixed.

FIG. 12 is an external drawing of a thoracotomy type sternum piercing tool.

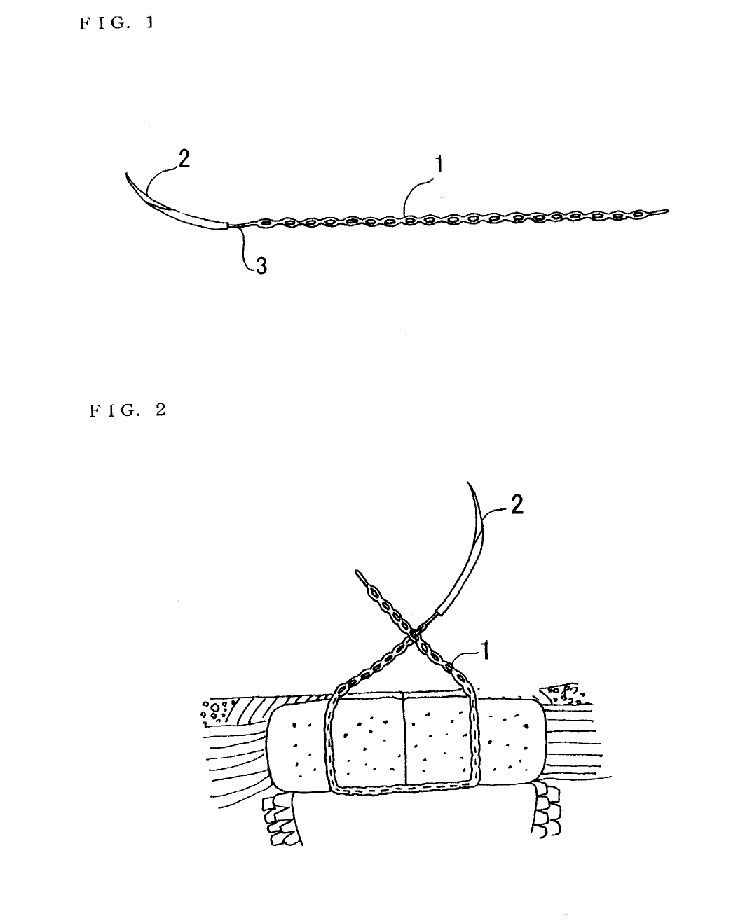

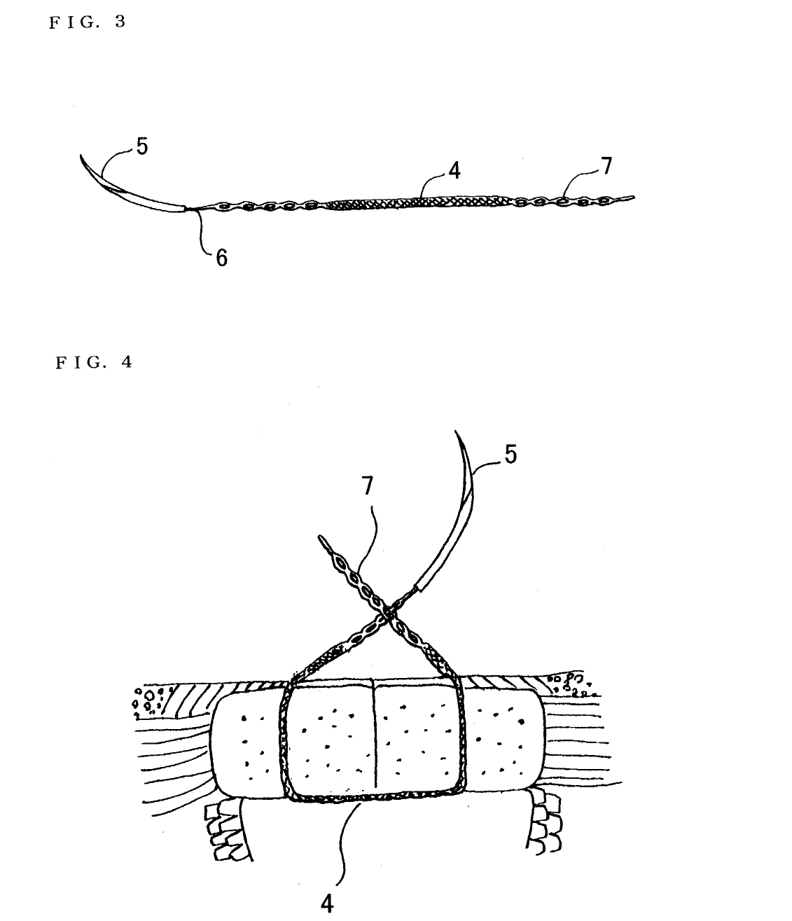

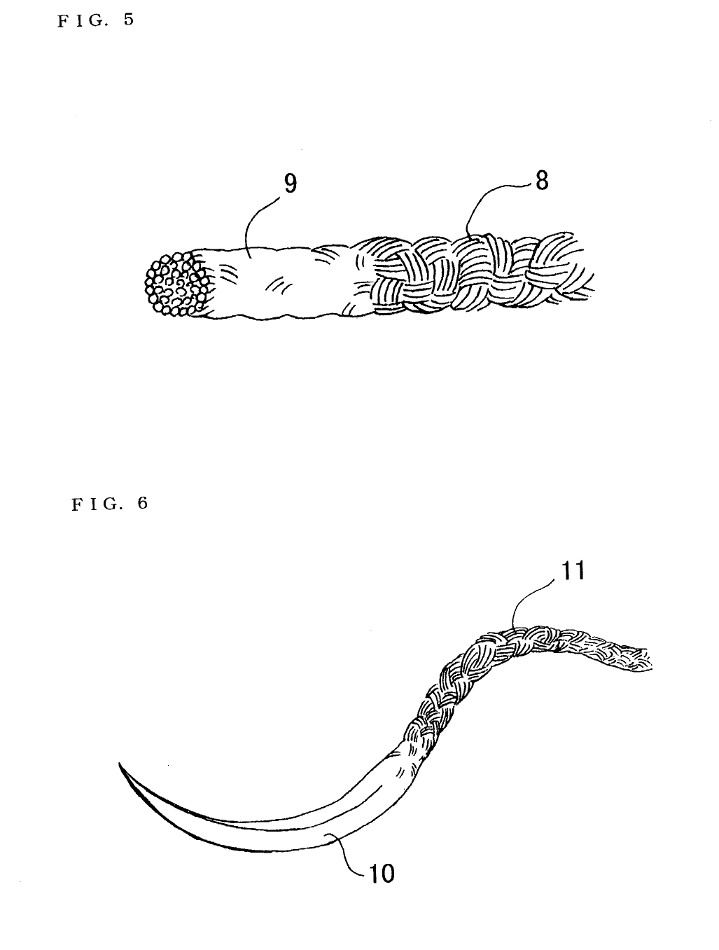

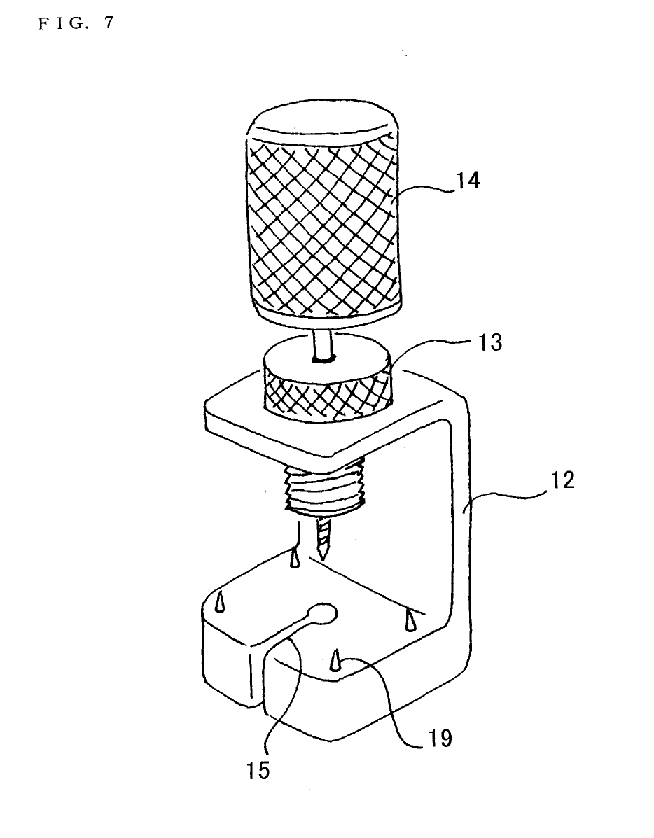

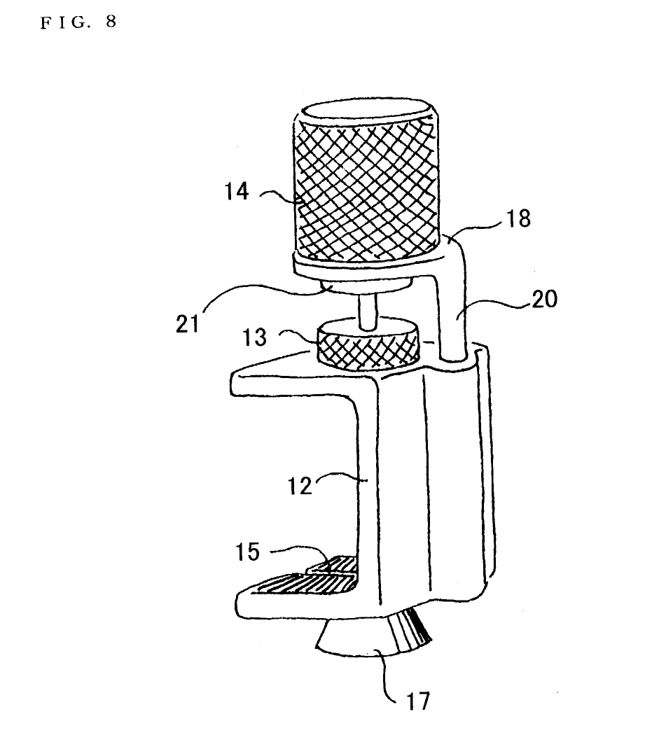

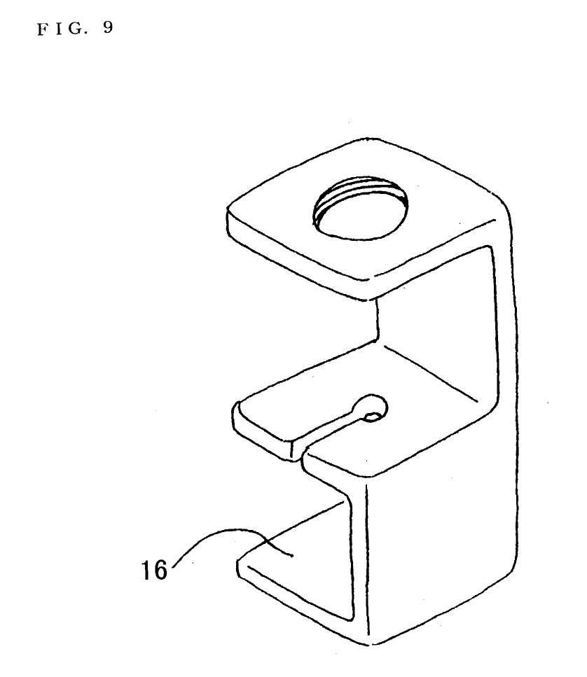

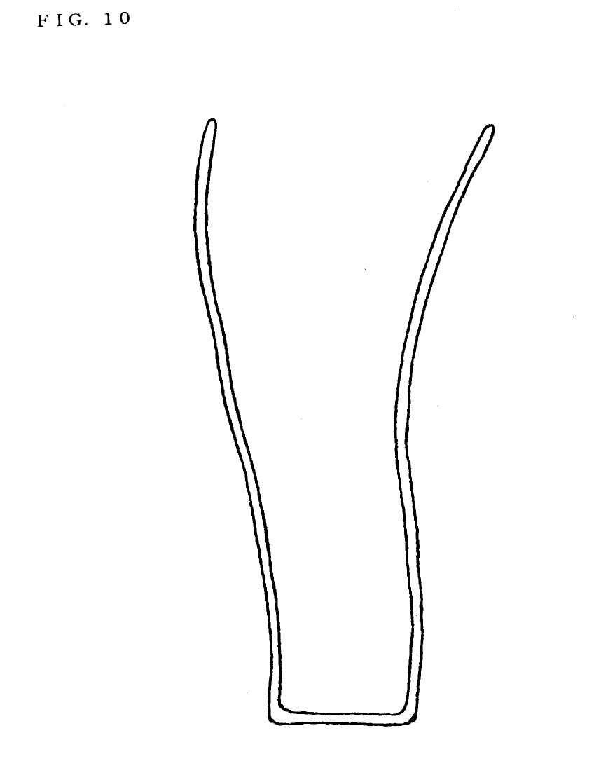

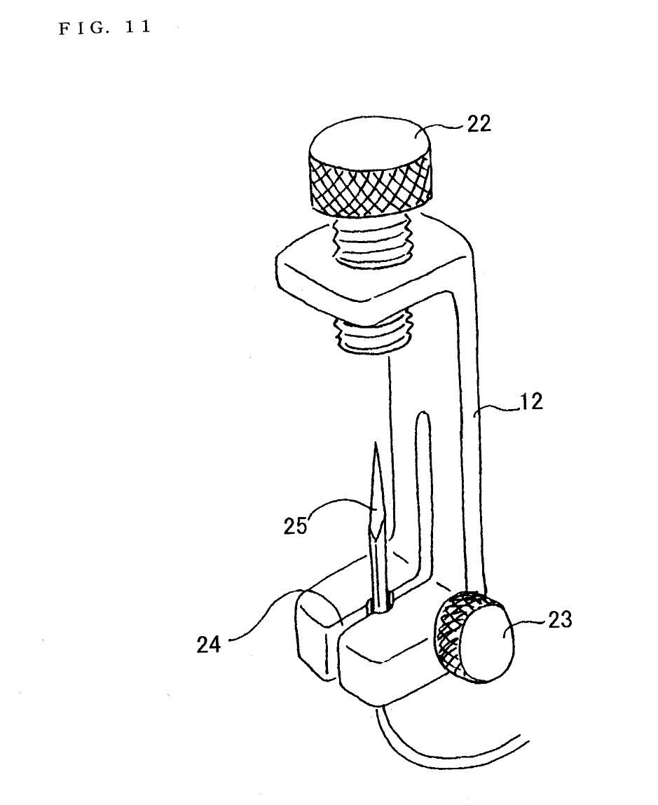

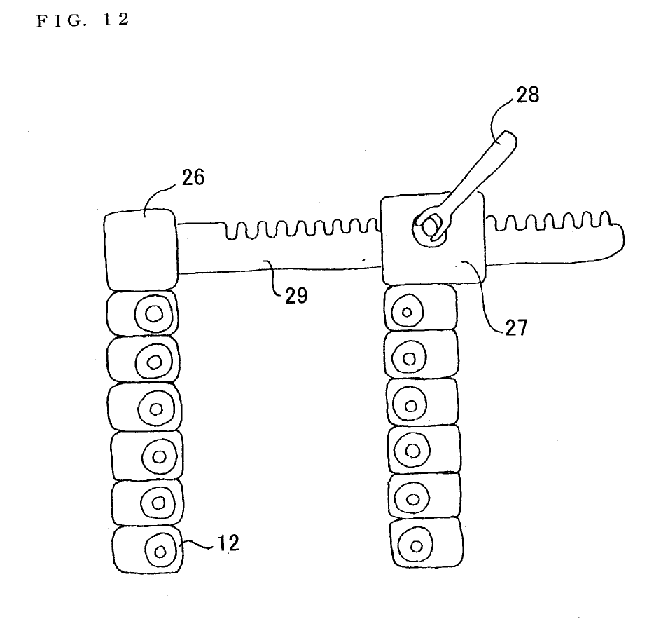

What is claimed is:

1. A sternum suture material used when closing the sternum during cardiac surgery, the material made of thin monofilaments or thin wire cross-woven by combining the monofilament and the wire and forming one loop or a multiple number of chain-like rings in the cable, the thread or the wire becomes a straight line during suture due to tension, the cable easy to fasten at time of fastening because of utilizing the rings of the chain, a straight needle or a curved needle to pierce the sternum is connected to one or both ends of the wire, the end parts of the wire to which the needle is connected is woven in a thinly narrowed way.

2. A sternum suture material as claimed in claim 1, wherein the center part is woven in a plain weave in a tape form, and the end parts to which the needles are connected are woven in a thinly narrowed manner.

3. A sternum suture material as claimed in claims 1 and 2, wherein the material is made of non-magnetic property substance.

4. A sternum suture material as claimed in claims 1 and 2, wherein the material is made of a substance that can be absorbed within the body.

5. A manufacturing method of the thread or the wire parts of the sternum suture material as claimed in claims 1 and 2, wherein an automatic weaving machine Torsion is utilized when weaving the thread or wire, forming one or a multiple number of rings or chain-like form by conducing uniform continuous weaving, the center part of the wire is made in plain weave in a tape form, the ends of the wire are woven in a thinly narrowed manner, a hole is made in the base part of a needle in a vertical direction using a drill, laser or discharge processing, the end of the material is inserted into that hole, the circumference of the needle is compressed uniformly and the needle is fixed into the wire.

6. A manufacturing method of the thread of the wire parts of the sternum suture material as claimed in claims 1 and 2, wherein a braiding machine is used when weaving the thread or wire, forming one or a multiple number of rings or chain-like form by conducting uniform continuous weaving, the center part of the wire can be made in plain weave in a tape form, the ends of the wire are woven in a thinly narrowed manner, a hole is made in the base part of a needle in a vertical direction using a drill, laser or discharge processing, the end of the material is inserted into that hole, the circumference of the needle is compressed uniformly and the needle is fixed into the wire.

7. A manufacturing method of sternum suture metal cable with a needle attached, a manufacturing method of a metal cable used for sternum suture during cardiac surgery, wherein the center part of the cable is woven of thin metal monofilaments with a braiding machine or a Torsion, either one or both ends of the cable has flax applied, then silver wax is adhered by heating the wax with a burner thence harden the ends to facilitate the needle attachment for suture.

8. A sternum suture metal cable used during cardiac surgery, wherein the center part of the cable is woven of thin metal monofilaments with a braiding machine or a Torsion, either one or both ends of the cable has flax applied, then silver wax is adhered by heating the wax with a burner thence harden the thin metal monofilaments, and furthermore process the ends into a straight or a curved needle with a pressing machine, the end of the cable itself is made into a needle to pierce through the sternum directly.

9. A sternum suture metal cable as claimed in claim 8 wherein the needle at the end of the cable is made as a dull-end needle.

10. A sternum piercing tool utilized for sternum suture during cardiac surgery, consisting of three parts being sternum fixing hook, adjusting screw, and piercing tool, wherein a thread cutting hole is made at the upper part of a U-shaped sternum fixing hook for the adjusting screw to be inserted, so when the adjusting screw is rotated the sternum is pressed and fixed between the base part of the sternum fixing hook and the adjusting screw, a hole for the piercing tool is made in a vertical direction at the center of the adjusting screw, the base part of the sternum fixing hook has a hole in the direction of the needlepoint of the piercing tool, and that hole is continued onto the side slit from which the sternum suture material are pulled out after being passed through the sternum, and side ditches or spikes and such are applied at the top part of the base portion of the sternum fixing hook for slip-proof of the sternum, when the sternum if pressed and fixed between the base part of the sternum fixing hook and the adjusting screw, a hole is pierced into the sternum with the piercing tool from the hole at the top of the adjusting screw, the hole through which sternum suture material are passed through to the sternum, and then the suture is conducted.

11. A sternum piercing tool as claimed in claim 10, wherein the sternum fixing hook has a vertical hole on its back side in the center, the columnar shaft is inserted to that hole, and one end of the projected columnar shaft is bent perpendicularly and is continued to an elevation table, and the elevation table is united with the piercing tool grip part sandwiched with a fixing plate, the elevation table is rotated having the columnar shaft as the axis, the needlepoint of the piercing tool is matched with the position of the hole, and the columnar shaft is made to have the function as the guide when the piercing tool is lowered down.

12. A sternum piercing tool as claimed in claims 10 and 11, the thickness of the lower part of the sternum fixing hook is made so that the needlepoint of the piercing tool digs into the top end of the base side of the sternum fixing hook when the piercing tool is pierced into the sternum in cases where the height of the inside of the sternum fixing hook is the same as the thickness of the sternum, and so that the piercing tool needlepoint does not go beyond the base side of the lower part of the sternum fixing hook when the piercing tool needlepoint is pierced into the sternum in cases where the sternum is thin after screwing in the adjusting screw to its maximum.

13. A sternum piercing tool used for sternum suture during cardiac surgery, which consists of three parts being the sternum fixing hook, adjusting screw, and piercing tool, wherein a thread cutting hole is made at the upper part of a U-shaped sternum fixing hook for the adjusting screw to be inserted, so when the adjusting screw is rotated the sternum is pressed and fixed between the base part of the sternum fixing hook and the adjusting screw, a hole for the piercing tool is made in a vertical direction at the center of the adjusting screw, the base part of the sternum fixing hook has a hole in the direction of the needlepoint of the piercing tool, and that hole is continued onto the side slit for which the sternum suture material are pulled out after being passed through the sternum, and side ditches or spikes and such are applied at the top part of the base portion of the sternum fixing hook for slip-proof of the sternum, the hole at the base portion of the sternum fixing hook is a piercing hole expanding in the lower part in a cone shape facilitating the passing through of the sternum suture material from below, the sternum is pressed and fixed between the base part of the sternum fixing hook and the adjusting screw, a hole is pierced onto the sternum with the piercing tool from the hole at the top of the adjusting screw, the hole through which sternum suture material is passed through to the sternum, and then the suture is conducted.

14. A sternum piercing tool as claimed in claim 13 wherein the sternum fixing hook is structured so that a plate is continued from the lower part to the front side or to the back side vertically downwards, and the plate is bent perpendicularly once again to be in parallel to the lower part of the sternum fixing hook, to function as the protection plate to protect the lung from being damaged by the needlepoint of the piercing tool.

15. A sternum suture wire for suture of sternum used after a hole is pierced with the above sternum piercing tool, wherein the center part is bent in advance in a U-shape, the length of the bent center part is equal to the distance between the two holes on the sternum one on the right and the other on the left side, the holes made with the sternum piercing tool as claimed in claims 13 and 14, the two ends of the wire is inserted into the sternum from below to above the sternum for fastening.

16. A sternum piercing tool used for sternum suture during cardiac surgery, wherein suture material is connected to the end of the piercing tool as a united part, the connection part between the piercing tool and the suture material are pressed in the piercing tool fixing slit at the base part of the sternum fixing hook, it is then screwed and fixed from the side with fixing screw, a thread cutting hole is made at the top part of the sternum fixing hook to insert the pull-up-screw, there is a hole at the base of the pull-up-screw for the needlepoint of the piercing tool to enter, so when the pull-up-screw is rotated the sternum fixing hook is pulled up, the piercing tool pierces the sternum from the back, the needlepoint of the piercing tool digs into the base of the pull-up-screw, then the fixing screw is loosened and the sternum fixing hook is taken off, the needlepoint of the piercing tool is pulled up and then the suture is conducted.

17. A sternum suture wire with an attached needle used for sternum suture using the sternum piercing tool as claimed in claim 16, wherein a hole is made with a drill, discharge process or laser to make a hole in a vertical direction at the tip end of the straight needle having an appropriate length to pierce through the sternum, the needle is inserted to either one end or both ends of the wire, and connected with caulking processing to prevent gaps or differences between the needle circumference and the wire, the cross-sectional form of the needle is made to be in a triangular or square form using press processing to facilitate the needle to pierce through even hard sternum, the material of the wire is soft quality metal material aiming for less invasiveness and damage onto the sternum.

18. A sternum piercing tool wherein the sternum fixing hook as claimed in claims 10, 11, 12, 13, 14 and 16, wherein a ball joint is applied to the upper side of the sternum fixing hook, a multiple number of sternum fixing hooks are fixed continuously, forming a fixing arm and a movable arm to be applied to the incision side of the sternum when cutting open the sternum to the right and left, one end of the fixing arm is connected perpendicularly to one end of the rail, and one end of the movable arm is in a box form to wrap around the rail, wherein continuous ditches are applied to the side of the rail, two pins inside the box enter into the rail ditches to move the movable arm, then the pins are connected with a handle that is projected above the box, so when the handle is rotated the movable arm moves above the rail and thus the sternum incision sides open and close.
